(19) United States
(12) Patent Application Publication
(10) Pub. No.: US 2013/0218161 A1
(43) Pub. Date: Aug. 22, 2013

(54) METHOD AND APPARATUS FOR MATERIAL REMOVAL

(71) Applicants: Jason A. Bryan, Avon Lake, OH (US); Wael K. Barsoum, Bay Village, OH (US)

(72) Inventors: Jason A. Bryan, Avon Lake, OH (US); Wael K. Barsoum, Bay Village, OH (US)

(73) Assignee: THE CLEVELAND CLINIC FOUNDATION, Cleveland, OH (US)

(21) Appl. No.: 13/593,582

(22) Filed: Aug. 24, 2012

Related U.S. Application Data
(60) Provisional application No. 61/527,424, filed on Aug. 25, 2011.

(51) Int. Cl. A61B 17/16

(52) U.S. Cl. CPC A61B 17/16
USPC 606/80

(57) ABSTRACT

A rotary surgical tool includes a driving shaft. A cutting head, connected to the driving shaft, is configured to cut into a surface. Driving means, connected to the driving shaft, rotate the driving shaft and the cutting head. A depth-limiting feature includes an adjustable stud extending from one of the cutting head and the surface toward the other one of the cutting head and the surface. The stud has a protrusion length that is greater than and/or equal to the length of a desired amount of final penetration of the cutting head into the surface. An aperture, provided in the other one of the cutting head and the surface, has an aperture depth that is greater than and/or equal to the desired amount of final penetration of the cutting head into the surface. Interaction between the aperture and the stud limits longitudinal advancement of the cutting head into the surface.

### RELATED APPLICATION

This application claims priority from U.S. Provisional Application No. 61/527,424, filed 25 Aug. 2011, the subject matter of which is incorporated herein by reference in its entirety.

### TECHNICAL FIELD

The present invention relates to an apparatus and method of material removal and, more particularly, to a method and apparatus for removing material from a surface in a depth-controlled manner.

### BACKGROUND OF THE INVENTION

The hip joint is located where the upper end of the femur meets the acetabulum. The femur, or thigh bone, looks like a long stem with a ball on the end. The acetabulum is a socket or cup-like structure in the pelvis, or hip bone. This “ball and socket” arrangement allows a wide range of motion, including sitting, standing, walking, and other daily activities.

During hip replacement, the surgeon removes the diseased bone tissue and cartilage from the hip joint. The healthy parts of the hip are left intact. Then the surgeon replaces the head of the femur (the ball) and the acetabulum (the socket) with new, artificial prosthetic implant components. The new hip is made of materials that allow a natural, gliding motion of the joint. Hip replacement surgery usually lasts 2 to 3 hours.

Sometimes the surgeon will use a special glue, or cement, to bond the new parts of the hip joint to the existing, healthy bone. This is referred to as a “cemented” procedure. In an uncemented procedure, the artificial parts are made of porous material that allows the patient's own bone to grow into the pores and hold the new parts in place. Doctors sometimes use a “hybrid” replacement, which consists of a cemented femur part and an uncemented acetabular part.

In order to prepare the head of the femur and/or the acetabulum to accept the corresponding prosthetic implant component, the surgeon may machine the native bone tissue to remove irregularities or for any other desired reason. Commonly, any material-removal procedures are done in small intervals, using a sequence of similarly configured tools of slightly different sizes. For example, to hollow out an acetabulum, a series of reamers having similar profiles but differing sizes can be used sequentially. This serial machining process allows the surgeon to gradually approach the desired final machined contours/sizes in small steps, which may be desirable in avoiding over-machining and accidental removal of too much of the native bone tissue.

Particularly when preoperative planning has specified a desired location and depth of installation for the final positioning of the prosthetic implant component, the surgeon may have a need to monitor and/or control how deep a tool, such as a reamer, is permitted to penetrate into the native bone tissue during use.

### SUMMARY OF THE INVENTION

In an embodiment of the present invention, a rotary surgical tool is described. A driving shaft has longitudinally spaced first and second driving shaft ends. A cutting head is connected to the first driving shaft end and is configured to cut into a surface. Driving means are connected to the second driving shaft end to directly rotate the driving shaft and indirectly rotate the cutting head through connection via the driving shaft. A depth-limiting feature includes a stud extending from a chosen one of the cutting head and the surface toward the other one of the cutting head and the surface. The stud has a protrusion length that is at least one of greater than and equal to the length of a desired amount of final penetration of the cutting head into the surface. The stud is adjustable to adjust the limit of the longitudinal advancement of the cutting head into the surface. An aperture is provided in the other one of the cutting head and the surface. The aperture has an aperture depth that is at least one of greater than and equal to the desired amount of final penetration of the cutting head into the surface. Interaction between the aperture and the stud limits longitudinal advancement of the cutting head into the surface.

In an embodiment of the present invention, a method of removing material from a surface in a depth-controlled manner is described. A material-removal tool is provided. A stud extends from a chosen one of the material-removal tool and the surface toward the other one of the material-removal tool and the surface. An aperture is provided in the other one of the material-removal tool and the surface. The aperture is configured to have an aperture depth that is at least one of greater than and equal to the length of a desired final penetration of the material-removal tool into the surface. The stud is configured to have a protrusion length that is at least one of greater than and equal to the length of the desired final penetration of the material-removal tool into the surface. The aperture and stud are brought into engagement. The material-removal tool is advanced longitudinally toward the surface. The surface is contacted with the material-removal tool in a material-removing manner. The aperture and the stud are interacted to limit longitudinal advancement of the material-removal tool into the surface. At least one of the material-removal tool, the stud, and the aperture is selectively adjusted to adjust the limit of the longitudinal advancement of the material-removal tool into the surface.

In an embodiment of the present invention, a material-removal apparatus for selectively removing material from a surface is described. A material-removal head is provided. A stud extends from a chosen one of the material-removal head and the surface toward the other one of the material-removal head and the surface. The stud has a protrusion length which is at least one of greater than and equal to the length of a desired final penetration of the material-removal head into the surface. The stud is selectively adjustable to adjust the limit of the longitudinal advancement of the material-removal head into the surface. An aperture is provided in the other one of the material-removal head and the surface. The aperture has an aperture depth which is at least one of greater than and equal to the length of the desired final penetration of the material-removal head into the surface. A user interface is located opposite the material-removal head from at least one of the stud and the aperture. Interaction between the aperture and the stud limits longitudinal advancement of the material-removal head into the surface.

### BRIEF DESCRIPTION OF THE DRAWINGS

For a better understanding of the invention, reference may be made to the accompanying drawings, in which:

FIG. 1 is a schematic side view of an embodiment of the present invention;

FIG. 2 is a perspective side view of a component of the embodiment of FIG. 1;

FIGS. 3A-3C are schematic side views of optional components of the present invention for use with the component of FIG. 2;

FIGS. 4A-4C are schematic side views depicting a sequence of operation of an embodiment of the present invention;

FIGS. 5A-5C are schematic side views of a series of configurations of a component of the present invention for use with the embodiment of FIGS. 4A-4C;

FIG. 6 is a schematic side view of a component of the present invention;

FIGS. 7A-7C are schematic side views of optional components of the present invention for use with the component of FIG. 6;

FIGS. 8A-8C are schematic side views depicting a sequence of operation of an embodiment of the present invention;

FIG. 9 is a schematic side view of an embodiment of the present invention;

FIG. 10 is a schematic side view of an embodiment of the present invention;

FIG. 11 is a schematic side view of a use environment for the present invention;

FIG. 12 is a perspective side view of a use environment for the present invention; and

FIG. 13 is a schematic top view of a use environment for the present invention.

### DESCRIPTION OF EMBODIMENTS

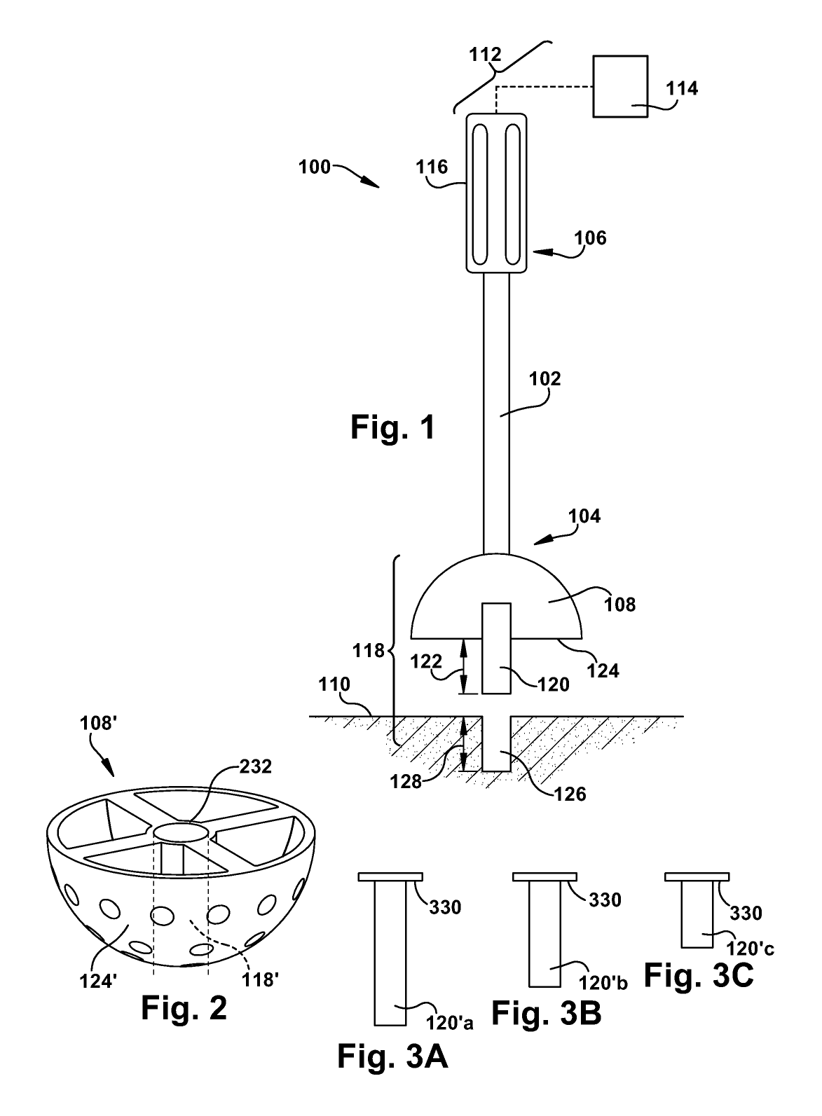

In accordance with the present invention, FIG. 1 depicts a material-removal apparatus 100, shown here as a rotary surgical tool, such as, but not limited to, a miller or reamer. Any number of non-rotary surgical tools could also or instead be used with the present invention.

As depicted in FIG. 1, the apparatus 100 includes a driving shaft 102 having longitudinally spaced first and second driving shaft ends 104 and 106, respectively. A material-removal head, such as cutting head 108, is connected to the first driving shaft end 104 and is configured to selectively cut into a surface 110. Any suitable type and/or combination of driving means 112 are connected to any suitable portion of the apparatus 100, but are shown here as being connected to the second driving shaft end 106 to directly rotate the driving shaft 102 and indirectly rotate the cutting head 108 through connection via the driving shaft. Two example driving means 112 are shown in FIG. 1: a schematic driving motor (pneumatic, hydraulic, electric, or any other suitable type) 114 is shown as being connected to the second driving shaft end 106; and a user-manipulable handle 116, which may be manually turned or otherwise manipulated by a user, is also shown as being connected to the second driving shaft end 106—both of these example driving means may be used singly or in combination with each other or with any other desired driving means. It is also contemplated that the apparatus 100 may have a user interface (e.g., a user-manipulable handle 116, when present, or simply the second driving shaft end 106) which is configured for receipt by a chuck of a driving tool or other driving means.

The surface 110 may be any suitable surface, though is described herein as being a patient tissue, such as, but not limited to, at least one of an acetabular surface, a femoral head surface, a glenoid surface, a humeral head surface, or any other patient bone surface. One of ordinary skill in the art can readily provide a material-removal tool having a desired shape, size, configuration, sharpness, material-removing action, or any other physical properties for use with a particular type of surface 110.

A depth-limiting feature 118 is provided to the apparatus 100. The depth-limiting feature 118 includes a stud 120 which extends from a chosen one of the cutting head 108 and the surface 110 toward the other one of the cutting head and the surface. Here, the stud 120 is shown as extending from the cutting head 108. The stud has a protrusion length 122 (i.e., the length which protrudes from a leading surface 124 of the cutting head 108) which is greater than or equal to a desired amount of final penetration of the cutting head into the surface 110. The stud 120 may be selectively adjustable to adjust the limit of longitudinal advancement of the cutting head 108 into the surface. (Here, “longitudinal” is used to mean a direction generally toward the top or bottom of the page, in the orientation of FIG. 1).

The depth-limiting feature 118 also includes an aperture 126 in the other one of the cutting head 108 and the surface 110 (as shown in FIG. 1, the aperture bores into the surface). The aperture 126 has an aperture depth 128 which is greater than or equal to the desired amount of final penetration of the cutting head 108 into the surface 110. The aperture 126 may be selectively adjustable to adjust the limit of longitudinal advancement of the cutting head 108 into the surface.

Optionally, the user interface (e.g., the driving means 112) may be located on an opposite side of the cutting head 108 from the stud 120 and/or the aperture 126.

It will be understood that the “desired amount of final penetration of the cutting head 108 into the surface 110″ is based upon the penetration at or near the depth-limiting feature 118, and that other portions of the cutting head 108 may penetrate at different depths into the surface 110, based upon the design of the cutting head.

As will be described in more detail below, interaction between the aperture 126 and the stud 120 limits longitudinal advancement of the cutting head 108 into the surface 110. For example, the stud 120 of the cutting head 108 shown in FIG. 1 will “bottom out” in the aperture 126 and prevent the cutting head 108 from advancing further into the surface 110 once the desired depth is reached. The apparatus 100 may be configured so that the same cutting head 108 is used, optionally in a substantially continuous manner (i.e., no significant stoppage or withdrawal of the cutting head 108 during its travel from initial contact with the surface 110 through reaching the desired final depth). However, it is also contemplated that at least one of the cutting head 108, the stud 120, and/or the aperture 126 may be selectively adjusted to provide deeper longitudinal advancement of the cutting head 108 into the surface. In the latter situation, the travel of the cutting head 108 from initial contact with the surface 110 through reaching the desired final depth could be made as a series of smaller, intermediate material-removal steps. This may be useful, for example, when the total volume of material to be removed is large enough that a better outcome is predicted via a series of small removals rather than one large removal, when the material-removal resolution needed is very fine (and perhaps unknown at the start of the process), when a finer-grain “polish” is desired for the final cut surface than the initial cutting head 108 would provide, or for any other desired reason.

In use, the aperture 126 of FIG. 1 is first generated in the surface 110 by any suitable means and at any desired location. It is contemplated that the depth of the aperture 126 into the surface 110 will be controlled to have some relationship (e.g., direct proportionality) to the amount of material which the cutting head 108 is to remove from the surface. For example, in many use environments, the aperture 126 will have a depth greater than or equal to the desired longitudinal travel of the cutting head 108 into the surface 110. The stud 120 length may also or instead be chosen to have some relationship (e.g., direct proportionality) to the amount of material which the cutting head 108 is to remove from the surface. For example, in many use environments, the stud 120 will have a length greater than or equal to the desired longitudinal travel of the cutting head 108 into the surface 110.

Optionally, the position of at least one of the stud 120 and the aperture 126 may be used to laterally (i.e., perpendicular to the longitudinal direction) guide positioning of the cutting head 108 with respect to the surface 110. For example, and particularly when at least a portion of the apparatus 100 blocks the user's view of the surface 110, the user could place the stud 120 lightly upon the surface 110 at the approximate estimated position of the aperture 126 and then—again, lightly—drag the stud across the surface until it at least partially “falls” into the aperture, thus indicating that the desired positioning of the cutting head 108 with respect to the surface 110 has been achieved. It is contemplated that, for many use applications of the present invention, this guiding function alone will not mark, scar, or otherwise materially alter the surface 110.

Once the cutting head 108 has been placed in the desired lateral location with respect to the surface 110 (regardless of how this happens), the apparatus 100 of FIG. 1 is used by advancing the apparatus longitudinally toward the surface until the aperture 126 and stud 120 are brought into operative engagement (whether or not actual contact between the two occurs). As shown in FIG. 1, at least a lower portion of the stud 120 may enter the aperture 126 in a male-to-female manner.

The cutting head 108 contacts the surface 110 in a material-removing manner. For example, the cutting head 108 may be manually or automatically rotated to bring a “grating” or “shaving” feature of the leading surface 124 into contact with the surface 110. As the cutting head 108 “bites” into the surface and removes material, the aperture 126 and stud 120 interact—here, by the stud entering further into the aperture as the surface 110 is ablated/eroded or otherwise machined away by the cutting head.

When the stud 120 “bottoms out” in the aperture 126, the interaction between the two limits longitudinal advancement of the cutting head 108 into the surface 110. The user may feel this interaction between the stud 120 and the aperture 126 as a “hard stop”, and/or some automatic or manual means may be provided to alert the user that the stud 120 has reached its maximum travel depth with respect to the aperture 126. For example, contact between the stud 120 and aperture 126 could complete an electrical circuit to cause a “stop” signal to be provided to the user, a mechanical and/or electric “circuit breaker” (e.g., a load cell) could provide feedback to the apparatus 100 and/or the user to indicate that additional longitudinal travel of the cutting head 108 is undesirable, or any other alert may be provided to the user and/or used to impede further operation of the cutting head 108.

The user then can remove the apparatus 100 from the surface 110 and, optionally, selectively adjust the cutting head 108, stud 120, and/or aperture 126 to adjust the limit of the longitudinal advancement of the cutting head into the surface. (For example, the aperture 126 could be lengthened and the above procedure repeated if the user would like to remove more material from the surface 110.) Once the user is satisfied with the material-removal procedure, the apparatus 100 is removed from the surface 110 vicinity, optionally the aperture 126 (which may be diminished in depth due to removal of surrounding material) may undergo further machining or even filling processes, and the user can proceed with any further tasks at/near the surface 110.

FIGS. 2-4C illustrate a second embodiment of an apparatus 100′. The apparatus 100′ of FIGS. 2-4C is similar to the apparatus of FIG. 1 and therefore, structures of FIGS. 2-4C that are the same as or similar to those described with reference to FIG. 1 have the same reference numbers with the addition of a “prime” mark. Description of common elements and operation similar to those in the previously described embodiments will not be repeated with respect to the second embodiment.

Turning to FIG. 2, an example of a suitable cutting head 108′ component is shown as a reamer head having an at-least-partially-spherical convex leading surface 124′ for use with a surface 110′ which is concave, like an acetabulum. When a FIG. 2 reamer-style cutting head 108′ is used, material is removed along a convex profile which substantially mates with the concave surface 110′, which itself is concave either before or as a result of the material-removal procedure. At least a portion of a depth-limiting feature 118′ is shown in dotted line as extending down an axis of the cutting head 108′.

The depth-limiting feature 118′ for use with the cutting head 108′ of FIG. 2 is of the “sequential” or “serial” variety, wherein the material removal is accomplished in stages. In order to facilitate this stepwise procedure, a series of studs 120′a, 120′b, and 120′c (shown in FIGS. 3A-3C, respectively) are provided for use with the cutting head 108′ of FIG. 2.

The stud(s) 120′a, 120′b, 120′c may be operatively coupled to the cutting head 108′ in any suitable manner. As shown in FIGS. 2 and 3A-3C, each stud 120′a, 120′b, 120′c has a collar 330 which can interact with frame 232 of the cutting head 108′ and, optionally, a driving shaft (omitted from these Figures) to allow the stud 120′a, 120′b, 120′c to protrude from the leading surface 124′ of the cutting head while being held securely enough in a substantially stable position relative to the cutting head to limit a distance of longitudinal advancement of the cutting head into the surface 110′.

In use, the apparatus 100′ of the second embodiment is used much like that of the first embodiment. The stud 120′a of FIG. 3a is engaged with the cutting head 108′ in any suitable manner. For example, in the depicted arrangement, the stud 120′a, which is a first stud, is slid longitudinally downward into the frame 232 of the cutting head 108 until prevented from falling through by interaction between the collar 330 and the frame 232. The collar 330 and frame 232—or any other retaining mechanism provided—may hold the stud 120′a and cutting head 108′ together and prevent them from relative longitudinal motion in any suitable manner. For example, the collar 330 and frame 232 could be a “bayonet” type connection, the collar 330 could be sandwiched between the frame 232 and at least a portion of the driving shaft, or any other means could be used to provide the desired fixation.

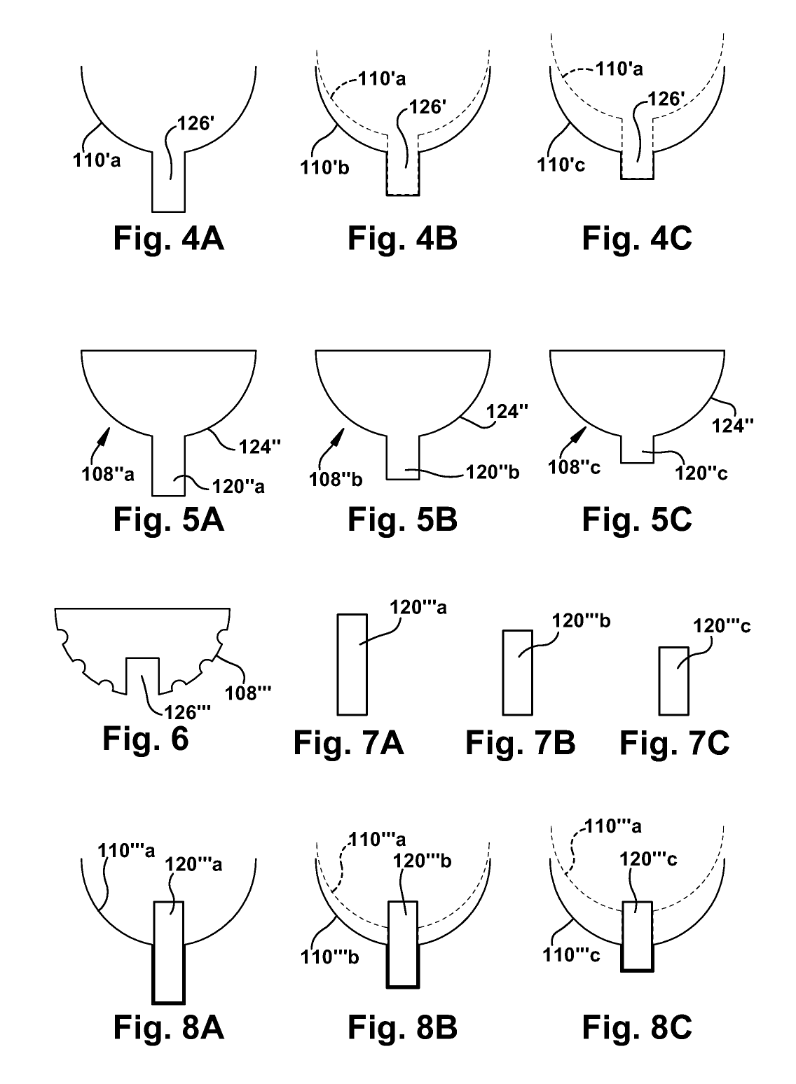

Once the stud 120′a is placed in the desired relationship with the cutting head 108′, the apparatus 100′ is placed into position longitudinally above the surface 110′ as desired. Here, FIG. 4A shows a concave surface 110′a before material removal has taken place. The apparatus 100′ is operated, as previously described, to remove material until the original surface 110′a has been reduced to intermediate surface 110′b, shown in FIG. 4B. In FIG. 4B, the first stud 120′a has interacted with the aperture 126′ to limit a first distance of longitudinal advancement of the cutting head 108′ into the surface 110′a.

As desired, a second stud 120′b, shown in FIG. 3B, having at least one physical dimension which differs from a corresponding physical dimension of the first stud 120′a, may be provided. In the depicted example, the second stud 120′b may be shorter than the first stud 120′a. The first stud 120′a may be removed from the cutting head 108′ and selectively replaced with the second stud 120′b. The apparatus 100′, with second stud 120′b, is then operated as previously described, but now is permitted to travel further toward the surface 110′a. Accordingly, with (shorter) second stud 120′b, the cutting head 108′ can longitudinally advance a second, greater distance into the surface 110′a than was permitted by the first stud 120′a, until sufficient material has been removed from the surface to cause the second stud 120′b to interact with the aperture 126 and limit further longitudinal travel of the cutting head into the surface. Using the second stud 120′b, the cutting head 108′ is permitted sufficient material removal to achieve the final surface 110′c, as shown in FIG. 4C.

Optionally, if the user wishes to remove material beyond final surface 110′c, any number of additional studs 120′ may be provided and used in a similar manner to the first and second studs 120′a and 120′b. For example, and as shown in FIG. 3C, third stud 120′c is shorter than either of the first and second studs 120′a and 120′b and thus will permit additional material removal. When a range or array of depth-limiting features 118′ (stud 120′ and/or aperture 126′ sizes) are provided, the “steps” in the range may allow stages of material removal at any desired resolution or depth intervals, and successive stages need not “match” previous stages in any physical extent. In other words, and as an example, the distance between surfaces 110′a and 110′b need not be the same as the distance between surfaces 110′b and 110′c.

Though the Figures of the present application are not to scale, it can be seen in FIGS. 4A-4C that the lateral dimensions of surfaces 110′a, 110′b, and 110′c (in addition to the longitudinal depths of these surfaces) are expanding via material removal during the process of operation of the apparatus 100′ in the sequence of FIGS. 4A-4C. Optionally, though not shown here, the same stud 120′ may be used, with different sized and/or shaped cutting heads 108′, to effectively adjust the protrusion length of the stud from the cutting head and achieve a substantially similar result to the FIGS. 4A-4C sequence of material removal. In other words, a first cutting head 108′ may be selectively replaced with a second cutting head having at least one physical dimension which differs from a corresponding physical dimension of the first cutting head—whether or not the stud 120′ and/or aperture 126′ are changed—to achieve a desired material-removal procedure. For example, to produce the laterally-widening surfaces 110′a, 110′b, and 110′c of FIGS. 4A-4C (whether or not the surfaces 110′a, 110′b, and 110′c also increase in depth), the series of cutting heads 108′ may have successively laterally wider leading surfaces 124′. One of ordinary skill in the art, with knowledge of the present invention, should be able to provide a range or sequence of studs 120′ and/or cutting heads 108′ to be used successively for a particular application of the present invention.

FIGS. 5A-5C illustrate a third embodiment of an apparatus 100″. The apparatus 100″ of FIGS. 5A-5C is similar to the apparatus of FIGS. 2-4C and therefore, structures of FIGS. 5A-5C that are the same as or similar to those described with reference to FIGS. 2-4C have the same reference numbers with the addition of a double “prime” mark. Description of common elements and operation similar to those in the previously described embodiments will not be repeated with respect to the third embodiment.

As alluded to above, the cutting heads 108″a, 108″b, and 108″c differ from each other in at least one physical dimension. Here, each cutting head 108″a, 108″b, and 108″c has an integral stud 120″a, 120″b, and 120″c, respectively. While the leading surface dimensions 124″ of all three cutting heads 108″a, 108″b, and 108″c are similar, the stud lengths 120″a, 120″b, and 120″c get successively shorter across the range of cutting heads, as shown in FIGS. 5A-5C, and therefore will successively allow further penetration into a surface (not shown) having relatively constant-depth aperture (not shown).

Stated differently, after the apparatus 100″ is initially operated to limit a first distance of longitudinal penetration into the surface 110″, the first cutting head 108″a having the first stud 120″a (shown in FIG. 5A) may be replaced on the apparatus with the second cutting head 108″b having the second stud 120″b, the second stud being long enough to limit a second distance of longitudinal advancement of the second cutting head into the surface, the second distance of longitudinal advancement resulting in greater longitudinal penetration of the second cutting head into the surface than does the first distance.

FIGS. 6-8C illustrate a third embodiment of an apparatus 100′″. The apparatus 100′ of FIGS. 6-8C is similar to the apparatus of FIGS. 2-4C and therefore, structures of FIGS. 6-8C that are the same as or similar to those described with reference to FIGS. 2-4C have the same reference numbers with the addition of a triple “prime” mark. Description of common elements and operation similar to those in the previously described embodiments will not be repeated with respect to the third embodiment.

One notable difference between the third embodiment of the present invention and those previously described is that the stud 120′ of the third embodiment is replaceably held by the surface 110′″ and interacts with an aperture 126′″ on the cutting head 108′. As can be seen in the sequence of FIGS. 8A-8C, the first stud 120′″a (shown in FIG. 7A) is successively replaced with the second stud 120′″b (shown in FIG. 7B) and the third stud 120′″c (shown in FIG. 7C) to allow successively deeper material removal from the initial surface 110′a, through the intermediate surface 110′b, to the final surface 110′″c, and even beyond, as desired. Each stud 120′″a, 120′b, and 120′″c interacts with the aperture 126′″ to limit the longitudinal travel of the cutting head 108′″ as desired.

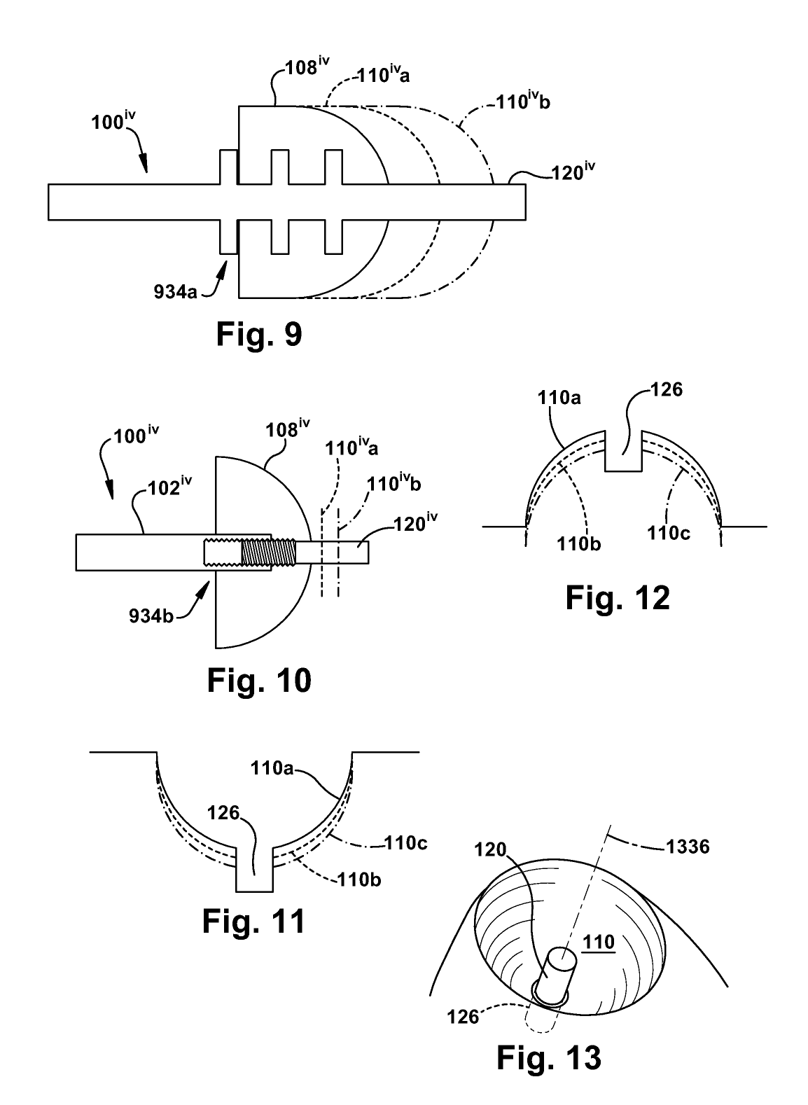

FIGS. 9-10 illustrate various example configurations of a fourth embodiment of an apparatus 100^{iv}. The apparatus 100^{iv} of FIGS. 9-10 is similar to the apparatus of FIG. 1 and therefore, structures of FIGS. 9-10 that are the same as or similar to those described with reference to FIG. 1 have the same reference numbers with the addition of a roman numeral “iv” superscript. Description of common elements and operation similar to those in the previously described embodiments will not be repeated with respect to the fourth embodiment.

In FIGS. 9-10, the stud 120^{iv} extends adjustably from either the cutting head 108^{iv} (the arrangement of these Figures) or the surface (not shown—this latter would be a similar arrangement to that of the third embodiment). As is apparent in FIGS. 9-10, each apparatus 100^{iv} has a different example style of stud adjustment means 934. The stud adjustment means 934a of FIG. 9 allows for a ratcheting motion of the cutting head 108^{iv} with respect to the stud 120^{iv}. The stud adjustment means 934b of FIG. 10 has the stud 120^{iv} threadably engaged with at least a portion of a driving shaft 102^{iv} which, itself, fixedly holds the cutting head 108^{iv} to permit relative adjustment of the stud with respect to the cutting head. Regardless of the use of these or any other configuration of stud adjustment means 934 for a particular embodiment of the present invention, the apparatus 100^{iv} is operated similarly to the above-described procedure to initially limit a first distance of longitudinal advancement of the cutting head 108^{iv} into the surface (this first distance shown in dotted line at 110^{iv} a in both FIGS. 9 and 10). Next, the stud 120^{iv} length is adjusted, using the stud adjustment means 934, with respect to at least one of the cutting head 108^{iv} and the surface 110^{iv}. The operation of the apparatus 100^{iv} is then repeated, with the at-least-partially-adjusted stud 120^{iv}, to limit a second distance of longitudinal advancement of the cutting head 108^{iv} into the surface (this second distance shown in dash-dot line at 110^{iv}b in both FIGS. 9 and 10). The second distance of longitudinal advancement results in greater longitudinal penetration of the cutting head 108^{iv} into the surface than does the first distance.

FIGS. 11-13 illustrate some general features of the surface 110′ which are common to all of the above-described embodiments, as well as to many other embodiments of the present invention.

FIGS. 11-12 show successive depths of material removal from the initial surface 110a, through the intermediate surface 110b, to the final surface 110c, for concave and convex surfaces, respectively. The concave arrangement of FIG. 11 has been described at length above. One of ordinary skill in the art will realize that an apparatus 100 could also or instead be configured for use with a convex surface (e.g., a femoral or humeral head) such that the cutting head 108 removes material along a concave-profiled leading surface 124 which substantially mates with a convex surface 110. Additionally or alternatively, an original native/virgin surface of any shape (flat, concave, convex, or any combination of these or other profiles) could be used with an apparatus 100 having a suitably configured cutting head 108 (e.g., one having a leading surface 124 which is flat, concave, convex, or any combination of these or other profiles) to impose a desired final surface/profile shape or configuration upon the original surface.

FIG. 13 is a perspective view of a concave surface (such as an acetabulum) which shows how a trajectory 1336 may be provided for material removal via an angular arrangement of at least one of the stud 120 and the aperture 126 with respect to the surface 110. In other words, the aperture 126 could be drilled and/or the stud 120 could be provided to the surface 110 at a desired trajectory 1336. The trajectory 1336 may be preoperatively or intraoperatively determined and may relate, for example, to a desired inclination and/or version of a to-be-installed prosthetic implant component (not shown) with respect to the surface 110. Through operation of the depth-limiting feature 118 during material removal by the apparatus 100, the material-removal depth and angulation with respect to the surface 110 could accordingly be guided along the trajectory by at least one of the aperture 126 and the stud 120.

While aspects of the present invention have been particularly shown and described with reference to the preferred embodiment above, it will be understood by those of ordinary skill in the art that various additional embodiments may be contemplated without departing from the spirit and scope of the present invention. For example, any of the described structures and components could be integrally formed as a single piece or made up of separate sub-components, with either of these formations involving any suitable stock or bespoke components and/or any suitable material or combinations of materials. It is contemplated that at least a portion of the apparatus 100 may be reusable (optionally sterilizable), and at least a portion of the apparatus may be disposable. Though certain components described herein are shown as having specific geometric shapes, all structures of the present invention may have any suitable shapes, sizes, configurations, relative relationships, cross-sectional areas, or any other physical characteristics as desirable for a particular application of the present invention. Any structures or features described with reference to one embodiment or configuration of the present invention could be provided, singly or in combination with other structures or features, to any other embodiment or configuration, as it would be impractical to describe each of the embodiments and configurations discussed herein as having all of the options discussed with respect to all of the other embodiments and configurations. A variety of schemes are described herein for placing the apparatus 100, or components thereof, into their predetermined position(s) with respect to the surface, and these schemes can be used singly or in any suitable combination for a particular application of the present invention. The mating relationships formed between the described structures need not keep the entirety of each of the “mating” surfaces in direct contact with each other but could include spacers or holdaways for partial direct contact, a liner or other intermediate member for indirect contact, or could even be approximated as desired with intervening space remaining therebetween and no contact. While the material-removal processes are generally characterized herein as being mechanical, rotary, blade-assisted processes (e.g., cutting or shearing), any other desired type of material-removal process, including, but not limited to, heat-based, chemical, abrasive, vacuum, other mechanical (including non-rotary), or any other type of material-removal scheme desired for a particular application of the present invention. A device or method incorporating any of these features should be understood to fall under the scope of the present invention as determined based upon the claims below and any equivalents thereof.

Other aspects, objects, and advantages of the present invention can be obtained from a study of the drawings, the disclosure, and the appended claims.

Having described the invention, we claim:

1. A rotary surgical tool, comprising:
a driving shaft having longitudinally spaced first and second driving shaft ends;
a cutting head, connected to the first driving shaft end and configured to cut into a surface;
driving means, connected to the second driving shaft end to directly rotate the driving shaft and indirectly rotate the cutting head through connection via the driving shaft; and
a depth-limiting feature, the depth-limiting feature comprising:
a stud extending from a chosen one of the cutting head and the surface toward the other one of the cutting head and the surface, the stud having a protrusion length that is at least one of greater than and equal to the length of a desired amount of final penetration of the cutting head into the surface, the stud being adjustable to adjust the limit of the longitudinal advancement of the cutting head into the surface;
an aperture in the other one of the cutting head and the surface, the aperture having an aperture depth that is at least one of greater than and equal to the desired amount of final penetration of the cutting head into the surface; and
wherein interaction between the aperture and the stud limits longitudinal advancement of the cutting head into the surface.

2. The rotary surgical tool of claim 1, wherein at least one of the cutting head, the stud, and the aperture is selectively adjusted to allow for deeper longitudinal advancement of the cutting head into the surface.

3. The rotary surgical tool of claim 2, wherein the stud is a first stud limiting a first distance of longitudinal advancement of the cutting head into the surface, and wherein the first stud is selectively replaced with a second stud, the second stud having at least one physical dimension which differs from a corresponding physical dimension of the first stud, the second stud limiting a second distance of longitudinal advancement of the cutting head into the surface, the second distance of longitudinal advancement resulting in greater longitudinal penetration of the cutting head into the surface than does the first distance.

4. The rotary surgical tool of claim 2, wherein the cutting head is a first cutting head, and wherein the first cutting head is selectively replaced with a second cutting head tool having at least one physical dimension which differs from a corresponding physical dimension of the first cutting head.

5. The rotary surgical tool of claim 2, wherein the stud extends adjustably from a chosen one of the cutting head and the surface to initially limit a first distance of longitudinal advancement of the cutting head into the surface;
wherein, after limiting the first distance of longitudinal advancement, the stud is at least partially adjusted with respect to the chosen one of the cutting head and the surface; and
wherein the aperture and the at-least-partially-adjusted stud interact to limit a second distance of longitudinal advancement of the cutting head into the surface, the second distance of longitudinal advancement resulting in greater longitudinal penetration of the cutting head into the surface than does the first distance.

6. The rotary surgical tool of claim 2, wherein the stud is a first stud and extends fixedly from the cutting head, which is a first cutting head, and wherein the first stud limits a first distance of longitudinal advancement of the cutting head into the surface;
wherein, after the cutting head has been limited to the first distance of longitudinal advancement into the surface, the first cutting head is replaced with a second cutting head having a second stud, the second stud having at least one physical dimension which differs from a corresponding physical dimension of the first stud, the second stud limiting a second distance of longitudinal advancement of the second cutting head into the surface, the second distance of longitudinal advancement resulting in greater longitudinal penetration of the second cutting head into the surface than does the first distance.

7. The rotary surgical tool of claim 1, wherein the cutting head is a reamer and the surface is at least one of an acetabular surface, a femoral head surface, a glenoid surface, and a humeral head surface.

8. The rotary surgical tool of claim 1, wherein the cutting head is a miller and the surface is a patient bone surface.

9. A method of removing material from a surface in a depth-controlled manner, the method comprising the steps of:
providing a material-removal tool;
providing a stud extending from a chosen one of the material-removal tool and the surface toward the other one of the material-removal tool and the surface;
providing an aperture in the other one of the material-removal tool and the surface;
configuring the aperture to have an aperture depth that is at least one of greater than and equal to the length of a desired final penetration of the material-removal tool into the surface;
configuring the stud to have a protrusion length that is at least one of greater than and equal to the length of the desired final penetration of the material-removal tool into the surface;
bringing the aperture and stud into engagement;
advancing the material-removal tool longitudinally toward the surface;
contacting the surface with the material-removal tool in a material-removing manner;
interacting the aperture and the stud to limit longitudinal advancement of the material-removal tool into the surface; and
selectively adjusting at least one of the material-removal tool, the stud, and the aperture to adjust the limit of the longitudinal advancement of the material-removal tool into the surface.

10. The method of claim 9, wherein the stud is a first stud;
wherein the step of interacting the aperture and the stud to limit longitudinal advancement of the material-removal tool into the surface includes the step of limiting a first distance of longitudinal advancement of the material-removal tool into the surface; and
wherein the step of selectively adjusting at least one of the material-removal tool, the stud, and the aperture includes the step of replacing the first stud with a second stud, the second stud having at least one physical dimension which differs from a corresponding physical dimension of the first stud;
the method including the step of interacting the aperture and the second stud to limit a second distance of longitudinal advancement of the material-removal tool into the surface, the second distance of longitudinal advancement resulting in greater longitudinal penetration of the material-removal tool into the surface than does the first distance.

11. The method of claim 9, wherein the stud extends adjustably from a chosen one of the material-removal tool and the surface, and wherein the step of interacting the aperture and the stud to limit longitudinal advancement of the material-removal tool into the surface includes the step of limiting a first distance of longitudinal advancement of the material-removal tool into the surface; the method including the steps of:
after limiting the first distance of longitudinal advancement, the stud is at least partially adjusted with respect to the chosen one of the material-removal tool and the surface; and
interacting the aperture and the at-least-partially-adjusted stud to limit a second distance of longitudinal advancement of the material-removal tool into the surface, the second distance of longitudinal advancement resulting in greater longitudinal penetration of the material-removal tool into the surface than does the first distance.

12. The method of claim 9, wherein the stud is a first stud and extends fixedly from the material-removal tool, which is a first material-removal tool;
wherein the step of interacting the aperture and the stud to limit longitudinal advancement of the material-removal tool into the surface includes the step of limiting a first distance of longitudinal advancement of the material-removal tool into the surface; and
wherein the step of selectively adjusting at least one of the material-removal tool, the stud, and the aperture includes the step of replacing the first material-removal tool with a second material-removal tool having a second stud, the second stud having at least one physical dimension which differs from a corresponding physical dimension of the first stud;
the method including the step of interacting the aperture and the second stud to limit a second distance of longitudinal advancement of the second material-removal tool into the surface, the second distance of longitudinal advancement resulting in greater longitudinal penetration of the second material-removal tool into the surface than does the first distance.

13. The method of claim 9, wherein the material-removal tool is a first material-removal tool, and wherein the step of selectively adjusting at least one of the material-removal tool, the stud, and the aperture to adjust the limit of the longitudinal advancement of the material-removal tool into the surface includes the step of replacing the first material-removal tool with a second material-removal tool having at least one physical dimension which differs from a corresponding physical dimension of the first material-removal tool.

14. The method of claim 9, wherein the material-removal tool is a rotary material-removal tool.

15. The method of claim 9, including the step of laterally guiding positioning of the material-removal tool with respect to the surface using the position of at least one of the stud and the aperture.

16. The method of claim 9, including the step of guiding a trajectory of insertion of the material-removal tool into the surface using the trajectory of at least one of the stud and the aperture with respect to the surface.

17. The method of claim 9, wherein the surface is convex and the material-removal tool removes material along a concave profile which substantially mates with the surface.

18. The method of claim 9, wherein the surface is concave and the material-removal tool removes material along a convex profile which substantially mates with the surface.

19. A material-removal apparatus for selectively removing material from a surface, the apparatus comprising:
a material-removal head;
a stud extending from a chosen one of the material-removal head and the surface toward the other one of the material-removal head and the surface, the stud having a protrusion length which is at least one of greater than and equal to the length of a desired final penetration of the material-removal head into the surface, the stud being selectively adjustable to adjust the limit of the longitudinal advancement of the material-removal head into the surface;
an aperture in the other one of the material-removal head and the surface, the aperture having an aperture depth which is at least one of greater than and equal to the length of the desired final penetration of the material-removal head into the surface; and
a user interface located opposite the material-removal head from at least one of the stud and the aperture;
wherein interaction between the aperture and the stud limits longitudinal advancement of the material-removal head into the surface.

20. The material-removal apparatus of claim 19, wherein at least one of the material-removal tool, the stud, and the aperture is selectively adjusted to provide deeper longitudinal advancement of the material-removal head into the surface.

21. The material-removal apparatus of claim 19, wherein the user interface is a user-manipulable handle.

22. The material-removal apparatus of claim 19, wherein the user interface is configured for receipt by a chuck of a driving tool.

23. The material-removal apparatus of claim 20, wherein the stud is a first stud limiting a first distance of longitudinal advancement of the material-removal head into the surface, and wherein the first stud is selectively replaced with a second stud, the second stud having at least one physical dimension which differs from a corresponding physical dimension of the first stud, the second stud limiting a second distance of longitudinal advancement of the material-removal head into the surface, the second distance of longitudinal advancement resulting in greater longitudinal penetration of the material-removal head into the surface than does the first distance.

24. The material-removal apparatus of claim 20, wherein the material-removal tool is a first material-removal tool, and wherein the first material-removal tool is selectively replaced with a second material-removal tool having at least one physical dimension which differs from a corresponding physical dimension of the first material-removal tool.

25. The material-removal apparatus of claim 20, wherein the stud extends adjustably from a chosen one of the material-removal head and the surface to initially limit a first distance of longitudinal advancement of the material-removal head into the surface;
wherein, after limiting the first distance of longitudinal advancement, the stud is at least partially adjusted with respect to the chosen one of the material-removal head and the surface; and
wherein the aperture and the at-least-partially-adjusted stud interact to limit a second distance of longitudinal advancement of the material-removal head into the surface, the second distance of longitudinal advancement resulting in greater longitudinal penetration of the material-removal head into the surface than does the first distance.

26. The material-removal apparatus of claim 20, wherein the stud is a first stud and extends fixedly from the material-removal head, which is a first material-removal head, and wherein the first stud limits a first distance of longitudinal advancement of the material-removal head into the surface;
wherein, after the material-removal head has been limited to the first distance of longitudinal advancement into the surface, the first material-removal head is replaced with a second material-removal head having a second stud, the second stud having at least one physical dimension which differs from a corresponding physical dimension of the first stud, the second stud limiting a second distance of longitudinal advancement of the second material-removal head into the surface, the second distance of longitudinal advancement resulting in greater longitudinal penetration of the second material-removal head into the surface than does the first distance.

27. The material-removal apparatus of claim 19, wherein the material-removal head is a rotary material-removal head.

28. The material-removal apparatus of claim 19, wherein positioning of the material-removal head is laterally guided with respect to the surface using the position of at least one of the stud and the aperture.

29. The material-removal apparatus of claim 19, wherein a trajectory of insertion of the material-removal head into the surface is guided using the trajectory of at least one of the stud and the aperture with respect to the surface.

30. The material-removal apparatus of claim 19, wherein the surface is convex and the material-removal head removes material along a concave profile which substantially mates with the surface.

31. The material-removal apparatus of claim 19 wherein the surface is concave and the material-removal head removes material along a convex profile which substantially mates with the surface.
